(19) United States
(12) Patent Application Publication
(10) Pub. No.: US 2022/0275105 A1
(43) Pub. Date: Sep. 1, 2022

(54) NEUTRALIZING GRANZYME B FOR PROVIDING CARDIOPROTECTION IN A SUBJECT WHO EXPERIENCED A MYOCARDIAL INFARCTION

(71) Applicants: INSERM (INSTITUT NATIONAL DE LA SANTÉ ET DE LA RECHERCHE MÉDICALE), Paris (FR); ASSISTANCE PUBLIQUE-HÔPITAUX DE PARIS (APHP), Paris (FR); SORBONNE UNIVERSITÉ, Paris (FR); UNIVERSITÉ DE PARIS, Paris (FR)

(72) Inventors: Hafid AIT-OUFELLA, Paris (FR); Nicolas DANCHIN, Paris (FR); Icia SANTOS ZAS, Paris (FR); Tabassome SIMON, Paris (FR)

(73) Assignees: INSERM (INSTITUT NATIONAL DE LA SANTÉ ET DE LA RECHERCHE MÉDICALE), Paris (FR); ASSISTANCE PUBLIQUE-HÔPITAUX DE PARIS (APHP), Paris (FR); SORBONNE UNIVERSITÉ, Paris (FR); UNIVERSITÉ DE PARIS, Paris (FR)

(21) Appl. No.: 17/632,271

(22) PCT Filed: Jul. 31, 2020
(86) PCT No.: PCT/EP2020/071626

§ 371 Date: Feb. 2, 2022

(30) Foreign Application Priority Data
Aug. 2, 2019 (EP) 19306002.7

(51) Int. Cl. C07K 16/40; A61P 9/00; A61K 31/713

(52) U.S. Cl. CPC C07K 16/40; A61P 9/00; A61K 31/713

(57) ABSTRACT

A method for providing cardioprotection in a subject who experienced a myocardial infarction, including administering a therapeutically effective amount of a Granzyme B inhibitor. Following acute MI in mice, CD8+ T lymphocytes are quickly recruited and activated in ischemic heart tissue, and release Granzyme B, leading to cardiomyocyte apoptosis and deterioration of myocardial function. Antibody-mediated depletion of CD8+ T lymphocytes decreases Granzyme B content and apoptotic within the myocardium and inflammatory response. mAb mediated-CD8 depletion limits myocardial injury and improves heart function. These effects are recapitulated in mice with CD8+ T cell selective Granzyme B deficiency in mice. Granzyme B is produced by other cell types (e.g., NK cells). Global Granzyme B deletion (GzmB^{-/-} mice) decreases apoptotic within the myocardium, reduces local pro-inflammatory signature and ultimately limits infarct size after MI. Elevated circulating levels of Granzyme B in patients with acute MI predict increased risk of death after 1 year.

### FIELD OF THE INVENTION

The present invention is in the field of medicine, and in particular in the field of cardiology.

### BACKGROUND OF THE INVENTION

Acute myocardial ischemia and reperfusion following primary PCI are responsible of cardiac tissue damages that lead to deleterious myocardial remodeling and heart failure. Many advances have been done in the early management of acute coronary thrombotic obstruction including rapid mechanical restauration of coronary artery blood flow and anti-platelet therapies [1]. In the context of myocardial infarction (MI), a progressive decline in early mortality over time has been observed in the United States [2] and Europe [3]. However, long term effects of ischemia-related cardiac damage remains a clinical and a social issue, including an increased risk of arrhythmia, heart failure and repetitive hospitalizations [4]. Thus, efforts have now to be directed towards targeting pathophysiological pathways involved in post-ischemic cardiac remodeling [5, 6].

There is a large body of human and experimental evidence showing that the immune response is involved in the long term cardiac complications of coronary occlusion [7]. In human and experimental myocardial infarction (MI), interruption of blood supply leads to rapid death of cardiac myocytes in the ischemic heart. Thereafter, inflammatory signals allow recruitment of inflammatory cells, which constitutes a major determinant of left ventricle (LV) remodelling, through their impact on extracellular matrix degradation/deposition, as well as on the clearance of dead cardiac myocytes and their debris. In mice, neutrophils massively infiltrate the myocardium within the first 24 hours, which is followed by a biphasic infiltration of two monocyte subsets (Ly6-C^{high} and Ly6C^{low}). Ly6-C^{high} monocytes dominate the acute phase of injury during the first 4 days and contribute to adverse tissue remodelling, while Ly6C^{low} monocytes become prevalent thereafter and are suggested to play a protective role in tissue healing and neovascularization [8]. CD4⁺ T cells infiltrate heart tissue within the first week following acute myocardial ischemia [9]. Resupplementation experiments showed that CD4⁺ T cells contribute to myocardial ischemia-reperfusion injury involving IFN-γ expression. A contrario, natural regulatory T cells (Tregs) protect against deleterious inflammatory remodelling following myocardial ischemia as Treg depletion using anti-CD25 antibody impaired left ventricular dilation and survival and expanding Tregs in vivo attenuates myocardial pro-inflammatory cytokine expression and leukocyte recruitment [10, 11]. TCR-independent [12] and -dependent mechanisms [13] have been identified in the activation of CD4³⁰ T cell subset following myocardial ischemia-reperfusion. Our group has shown that CCL-7 production by B cells at the acute phase of MI orchestrates monocyte mobilization and recruitment into the ischemic heart, with major impact on LV remodeling and function [14]. Recently, it has been suggested that depletion of CD8+ T cells would be suitable for the treatment of myocardial infarction (WO2017/064034). However, the mechanisms of CD8 mediated cardiac cytotoxicity remains unknown.

### SUMMARY OF THE INVENTION

As defined by the claims, the present invention relates to methods for providing cardioprotection in a subject who experienced a myocardial infarction.

### DETAILED DESCRIPTION OF THE INVENTION

Acute myocardial infarction (MI) is a common condition responsible for heart failure and sudden death. Here, the inventors show that following acute MI in mice, CD8⁺ T lymphocytes are quickly recruited and activated in the ischemic heart tissue, and release Granzyme B leading to cardiomyocyte apoptosis and deterioration of myocardial function. Antibody-mediated (CD8-specific antibody) depletion of CD8⁺ T lymphocytes decreases Granzyme B content and apoptotic within the myocardium and inflammatory response. Finally, CD8 depletion limits myocardial injury and improves heart function. These effects are recapitulated in mice with CD8⁺ T cell selective Granzyme B deficiency in mice. Granzyme B is also produced by other cell types such as NK cells. Interestingly, global Granzyme B deletion (GzmB^{-/-} mice) decreases apoptosis within the myocardium, reduces local pro-inflammatory signature and ultimately limits infarct size after MI. The inventors also show that elevated circulating levels of Granzyme B in patients with acute MI predict increased risk of death at 1-year follow-up. The work unravels a previously unsuspected pathogenic role of Granzyme B following acute ischemia, and identifies novel therapeutic targets for this devastating condition.

Accordingly, the first object of the present invention relates to a method for providing cardioprotection in a subject who experienced a myocardial infarction comprising administering the subject with a therapeutically effective amount of a Granzyme B inhibitor.

As used herein, the term “subject”, “individual,” or “patient” is used interchangeably and refers to any subject for whom diagnosis, treatment, or therapy is desired, particularly humans. Other subjects may include cattle, dogs, cats, guinea pigs, rabbits, rats, mice, horses, and the like. In some preferred embodiments, the subject is a human.

As used herein the term “cardioprotection” means protecting against or reducing damage to the myocardium after a myocardial infarction, after, during or prior to ischemic reperfusion. In particular, cardioprotection includes reducing infarct size, reducing ischemia-reperfusion injury, reducing hypoxia induced apoptosis/necrosis and preventing cardiomyocyte cell death. The method of the present invention is thus particularly suitable for the treatment of myocardial infarction injury in a subject in need thereof. More particularly, the method of the present invention is particularly suitable for reducing post ischemic left ventricular remodeling. More particularly, the method of the invention is suitable for increasing the left ventricle ejection fraction (LVEF), and/or for inhibiting left ventricle enlargement, and/or for reducing left ventricle end systolic volume, and/or reducing left ventricle end diastolic volume, and/or for ameliorating left ventricle dysfunction, and/or for improving myocardial contractibility.

The method of the present invention is suitable for reducing the risk or progression of heart failure. As used herein, the term “heart failure” or “HF has its general meaning in the art and embraces congestive heart failure and/or chronic heart failure. Functional classification of heart failure is generally done by the New York Heart Association Functional Classification (Criteria Committee, New York Heart Association. Diseases of the heart and blood vessels. Nomenclature and criteria for diagnosis, 6th ed. Boston: Little, Brown and co, 1964;114). This classification stages the severity of heart failure into 4 classes (I-IV). The classes (I-IV) are: Class I: no limitation is experienced in any activities; there are no symptoms from ordinary activities; Class II: slight, mild limitation of activity; the patient is comfortable at rest or with mild exertion;Class III: marked limitation of any activity; the patient is comfortable only at rest; Class IV: any physical activity brings on discomfort and symptoms occur at rest.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In some embodiments, the inhibitor of the present invention is administered to a subject having one or more signs or symptoms of acute myocardial infarction injury. In some embodiments, the subject has one or more signs or symptoms of myocardial infarction, such as chest pain described as a pressure sensation, fullness, or squeezing in the mid portion of the thorax; radiation of chest pain into the jaw or teeth, shoulder, arm, and/or back; dyspnea or shortness of breath; epigastric discomfort with or without nausea and vomiting; and diaphoresis or sweating.

In some embodiments, the inhibitor of the present invention is administered simultaneously or sequentially (i.e. before or after) with a revascularization procedure performed on the subject. In some embodiments, the subject is administered with the inhibitor of the present invention before, during, and after a revascularization procedure. In some embodiments, the subject is administered with the inhibitor of the present invention as a bolus dose immediately prior to the revascularization procedure. In some embodiments, the subject is administered with the inhibitor of the present invention continuously during and after the revascularization procedure. In some embodiments, the subject is administered with the inhibitor of the present invention for a time period selected from the group consisting of at least 3 hours after a revascularization procedure; at least 5 hours after a revascularization procedure;

at least 8 hours after a revascularization procedure; at least 12 hours after a revascularization procedure; at least 24 hours after a revascularization procedure. In some embodiments, the subject is administered with the inhibitor of the present invention in a time period selected from the group consisting of starting at least 8 hours before a revascularization procedure; starting at least 4 hours before a revascularization procedure; starting at least 2 hours before a revascularization procedure; starting at least 1 hour before a revascularization procedure; starting at least 30 minutes before a revascularization procedure. In some embodiments, the revascularization procedure is selected from the group consisting of percutaneous coronary intervention; balloon angioplasty; insertion of a bypass graft; insertion of a stent; directional coronary atherectomy; treatment with one or more thrombolytic agent(s); and removal of an occlusion.

As used herein, the term “Granzyme B” has its general meaning in the art and refers to an enzyme is necessary for target cell lysis in cell-mediated immune responses. For instance, Granzyme B cleaves caspase-3, -7, -9 and 10 to give rise to active enzymes mediating apoptosis. An exemplary amino acid sequence of Granzyme B is as set forth in SEQ ID NO:1.

>sp|P10144|GRAB_HUMAN Granzyme B OS = Homo sapiens
OX = 9606 GN = GZMB PE = 1 SV = 2
SEQ ID NO: 1
MQPILLLLAFLLLPRADAGEIIGGHEAKPHSRPYMAYLMIWDQKSLKRCGG
FLIRDDFVLTAAHCWGSSINVTLGAHNIKEQEPTQQFIPVKRPIPHPAYNP
KNFSNDIMLLQLERKAKRTRAVQPLRLPSNKAQVKPGQTCSVAGWGQTAPL
GKHSHTLQEVKMTVQEDRKCESDLRHYYDSTIELCVGDPEIKKTSFKGDSG
GPLVCNKVAQGIVSYGRNNGMPPRACTKVSSFVHWIKKTMKRY

As used herein, a “Granzyme B inhibitor” refers to any compound natural or not which is capable of inhibiting the activity or expression of Granzyme B. The term encompasses any Granzyme B inhibitor that is currently known in the art or that will be identified in the future, and includes any chemical entity that, upon administration to a patient, results in inhibition or down-regulation of a biological activity or expression of Granzyme B.

In some embodiments, the inhibitor of the present invention is an anti-Granzyme B neutralizing antibody.

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161 ; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001 ; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “nanobody®”. For a general description of (single) domain antibodies, reference is also made to the prior art cited above, as well as to EP 0 368 684, Ward et al. (Nature 1989 Oct 12; 341 (6242): 544-6), Holt et al., Trends Biotechnol., 2003, 21(11):484-490; and WO 06/030220, WO 06/003388.

As used herein, the term “neutralizing antibody” refers to an antibody that is capable of reducing or inhibiting (blocking) activity or signaling of the ligand as determined by in vivo or in vitro assays. Typically, the antibody of the present invention is capable of reducing and/or inhibiting the apoptosis of cardiomyocytes induced by Granzyme B.

In some embodiments, the antibody of the present invention is a single domain antibody. As used herein the term “single domain antibody” has its general meaning in the art and refers to the single heavy chain variable domain of antibodies of the type that can be found in Camelid mammals which are naturally devoid of light chains. Such single domain antibody are also “nanobody®”.

In some embodiments, the inhibitor of the present invention is an anti-Granzyme B monoclonal antibody.

Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In some embodiments, the antibody of the present invention is a fully human antibody. As used herein, the term “fully human” refers to an immunoglobulin, such as an antibody or antibody fragment, where the whole molecule is of human origin or consists of an amino acid sequence identical to a human form of the antibody or immunoglobulin. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

In some embodiments, the antibody of the present invention is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference.

In some embodiments, the inhibitor of the present invention is an aptamer. Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli Thioredoxin A that are selected from combinatorial libraries by two hybrid methods (Colas et al., 1996).

In some embodiments, the Granzyme B inhibitor is an inhibitor of Granzyme B expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of Granzyme B mRNA by binding thereto and thus preventing protein translation or increasing mRNA degradation, thus decreasing the level of Granzyme B, and thus activity, in a cell. For example, antisense oligonucleotides of at least about 15 bases and complementary to unique regions of the mRNA transcript sequence encoding Granzyme B can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. Granzyme B gene expression can be reduced by contacting a patient or cell with a small double stranded RNA (dsRNA), or a vector or construct causing the production of a small double stranded RNA, such that Granzyme B gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing Granzyme B. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. In some embodiments, the inhibitor of expression is an endonuclease. The term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR). In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences. In some embodiment, the endonuclease is CRISPR-cas9 which is from Streptococcus pyogenes. The CRISPR/Cas9 system has been described in US 8,697,359 B1 and US 2014/0068797. In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

By a “therapeutically effective amount” is meant a sufficient amount of the active ingredient for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

In some embodiments, the inhibitor of the present invention is administered in combination with an additional active agent. In some embodiments, the additional active agent is a cardiovascular agent selected from the group consisting of hyaluronidase, a corticosteroid, recombinant superoxide dismutase, prostacyclin, fluosol, magnesium, poloxamer 188, trimetazidine, eniporidine, cariporidine, a nitrate, anti-P selectin, an anti-CD18 antibody, adenosine, and glucose-insulin-potassium. In some embodiments, the cardiovascular agent is selected from the group consisting of an anti-arrhthymia agent, a vasodilator, an anti-anginal agent, a corticosteroid, a cardioglycoside, a diuretic, a sedative, an angiotensin converting enzyme (ACE) inhibitor, an angiotensin II antagonist, a thrombolytic agent, a calcium channel blocker, a throboxane receptor antagonist, a radical scavenger, an anti-platelet drug, a β-adrenaline receptor blocking drug, oreceptor blocking drug, a sympathetic nerve inhibitor, a digitalis formulation, an inotrope, and an antihyperlipidemic drug. In some embodiments, the active agent is an inotrope. Positive inotropic agents increase myocardial contractility, and are used to support cardiac function in conditions such as decompensated congestive heart failure, cardiogenic shock, septic shock, myocardial infarction, cardiomyopathy, etc. Examples of positive inotropic agents include, but are not limited to, Berberine, Bipyridine derivatives, Inamrinone, Milrinone, Calcium, Calcium sensitizers, Levosimendan, Cardiac glycosides, Digoxin, Catecholamines, Dopamine, Dobutamine, Dopexamine, Epinephrine (adrenaline), Isoprenaline (isoproterenol), Norepinephrine (noradrenaline), Eicosanoids, Prostaglandins, Phosphodiesterase inhibitors, Enoximone, Milrinone, Theophylline, and Glucagon. Negative inotropic agents decrease myocardial contractility, and are used to decrease cardiac workload in conditions such as angina. While negative inotropism may precipitate or exacerbate heart failure, certain beta blockers (e.g. carvedilol, bisoprolol and metoprolol) have been shown to reduce morbidity and mortality in congestive heart failure. Examples of negative inotropic agents include, but are not limited to, Beta blockers, Calcium channel blockers, Diltiazem, Verapamil, Clevidipine, Quinidine, Procainamide, disopyramide, and Flecainide. In some embodiments, the cardiovascular agent is cyclosporine. As used herein, the term “cyclosporine” refers to cyclosporine A, cyclosporine G, and functional derivatives or analogues thereof, e.g., NIM81 1. Cyclosporine A refers to the natural Tolypocladium inflation cyclic non-ribosomal peptide. Cyclosporine G differs from cyclosporine A in the amino acid 2 position, where an L-norvaline replaces the a-aniinobutyric acid. (See generally, Wenger, R. M. 1986. Synthesis of Ciclosporin and analogues: structural and conformational requirements for immunosuppressive activity. Progress in Allergy, 38:46-64).

Typically the active ingredient of the present invention (e.g. Granzyme B inhibitor) is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

A further object of the present invention relates to a method of screening a drug suitable for providing cardioprotection in a subject who experienced a myocardial infarction comprising i) providing a test compound and ii) determining the ability of said test compound to inhibit the expression or activity of Granzyme B.

Any biological assay well known in the art could be suitable for determining the ability of the test compound to inhibit the activity or expression of Granzyme B. In some embodiments, the assay first comprises determining the ability of the test compound to bind to Granzyme B. In some embodiments, a cardiomyocyte population is then contacted and activated so as to determine the ability of the test compound to inhibit the activity or expression of Granzyme B. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting the activity or expression of Granzyme B, as determined using in vitro methods described herein, are likely to exhibit similar modulatory capacity in applications in vivo. In vivo assays are well known in the art and typically include those described in the EXAMPLE. Typically, the test compound is selected from the group consisting of peptides, peptidomimetics, small organic molecules, antibodies (e.g. intraantibodies), aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1: Cytotoxic CD8⁺ T lymphocytes are activated and recruited to the ischemic tissue after myocardial infarction. mRNA levels of Granzyme B within the injured myocardium on day 1, day 3 and day 7 after coronary ligation or sham (n=8 mice per group/timepoint). *P<0.05 versus Sham.

FIG. 2. Global Granzyme B deficiency limits cardiac damage after acute MI.

(A) Acute MI was induced on C57bl6 Wild type (WT) mice or on Granzyme B deficiency (GzmB^{-/-}) mice. (B) Quantitative analysis (Left) of TUNEL+ cells in the peri-infarct area of WT C57BL/6J or GzmB^{-/-} mice at day 3 after MI (n=8-9 mice per group); *** P<0.001. (C) Il-1β, Il-6, Tnf-α, IL-10 and Mmp9 mRNA levels measured by qPCR in infarcted heart at Day 3 after MI, ** P<0.01, *** P<0.001. (D) Representative photomicrographs (Left) and quantitative analysis (Right) of infarct size evaluation evaluated by Masson trichrome staining, in the 2 groups of mice. (n=7 mice per group), * P<0.05.

FIG. 3. CD8⁺ T lymphocytes trigger adverse ventricular remodeling and alter heart function through the production of Granzyme B. (A) Rag1^{-/-} mice injected with either CD8-depleted splenocytes or CD8 cell-depleted splenocytes re-supplemented with wild-type or GzmB^{-/-} CD8+ T cells 3 weeks before MI. (B) Survival curves following MI (Pooled 3 experiments, n=10-18/group), *P<0.05, **P<0.01 and ***P<0.001. (C, D) Representative photomicrographs and quantitative analysis of infarct size (C), fibrosis and collagen content (D) in the 4 groups of mice, scale bar 100 μm. Results are pooled from three independent experiments with 7 to 9 mice per group. (E) Echocardiography analysis after 21 days of MI and assessment of LV shortening fraction (SF) in the 4 groups of mice. (F) Correlation between CD8+ T cell number in the spleen at day 21 and LV shortening fraction. Were included data from 2 groups: CD8-depleted splenocytes or CD8 cell-depleted splenocytes re-supplemented with wild-type CD8⁺ T cells.

FIG. 4. Survival according to baseline circulating Granzyme B level (< or > median value) in patients with acute MI. High level of Granzyme B at the admission for acute MI were independently predictive of death after one year of follow-up after multiple adjustments (see Methods). HR=Hazard ratio.

### EXAMPLE

Methods

Myocardial infarction. All mice were on full C57Bl/6J background. C57BL/6 (Janvier, France), Gzm-B^{-/-}, Rag1^{-/-} (Jackson, United States of America). Myocardial infarction was induced by left anterior descending coronary artery ligation [14]. Mice were anesthetized using ketamine (100 mg/kg) and xylazine (10 mg/kg) via intraperitoneal (i.p.) injection, then intubated and ventilated using a small animal respirator. The chest wall was shaved and a thoracotomy was performed in the fourth left intercostal space. The left ventricle was visualized, the pericardial sac was then removed and the left anterior descending artery was permanently ligated using a 7/0 non-absorbable monofilament suture (Peters surgical, France) at the site of its emergence under the left atrium. Significant color changes at the ischemic area were considered indicative of successful coronary occlusion. The thoracotomy was closed with 6/0 non-absorbable monofilament sutures (Peters surgical, France). The same procedure was performed for sham-operated control animals except that the ligature was left untied. The endotracheal tube was removed once spontaneous respiration resumed, and animals were placed on a warm pad maintained at 37° C. until the mice were completely awake.). Rag1^{-/-} mice received either 4×10⁶ CD8+ T cell-depleted splenocytes (Negative fraction using anti-CD8 beads), CD8+ T cell-depleted splenocytes re-supplemented with 6×10⁶ wild-type CD8+ T lymphocytes or with 6×10⁶ GzmB^{-/-} CD8+ T lymphocytes 21 days prior to myocardial infarction induction. Mice were then allowed to recover for 3 weeks Experiments were conducted according to the French veterinary guidelines and those formulated by the European Community for experimental animal use and were approved by the Institut National de la Santé et de la Recherche Médicale.

Echocardiographic measurements. Transthoracic echocardiography was performed at 9, 21 and 28 days after surgery using an echocardiograph (ACUSON S3000™ ultrasound, Siemens AG, Erlangen Germany) equipped with a 14-MHz linear transducer (1415SP). The investigator was blinded to group assignment. Animals were anesthetized by isoflurane inhalation. Two-dimensional parasternal long-axis views of the left ventricle were obtained for guided M-mode measurements of the LV internal diameter at end diastole (LVDD) and end systole (LVDS), as well as the interventricular septal wall thickness, and posterior wall thickness at the same points. Fractional shortening percentage (% FS) was calculated by the following formulas: % FS=[(LVDD−LVDS)/LVDD]X 100.

CD8+ T cell purification and transplantation. CD8+ T cells were isolated from C57BL/6J, Gzm-B^{-/-} spleens and purified using a CD8+ T cell isolation kit (Miltenyi Biotec; Paris, France) according to the manufacturer's protocol. In brief, CD8+ T cells were negatively selected using a cocktail of antibody-coated magnetic beads (CD4, CD11b, CD11c, CD19, CD45R (B220), CD49b (DX5), CD105, Anti-MHC-class II, Ter-119 and TCRγ/δ) followed by cell separation using LS magnetic columns (Miltenyi Biotec; Paris, France), yielding CD8+ T cells with >95% purity (Data not shown). Cells were then intravenously injected 21 days prior myocardial infarction in Rag1^{-/-} mice.

Histopathological and immunofluorescence analyses. Cardiac healing following myocardial infarction was assessed at day 21. Hearts were excised, rinsed in PBS and frozen in liquid nitrogen. Hearts were sectioned by a cryostat (CM 3050S, Leica) into 7-μm-thick sections. Masson's trichrome and Sirius Red stainings were performed for infarct size and myocardial fibrosis evaluation. Infarct size was calculated as a percentage of infarct area to total LV circumference. The collagen volume fraction was calculated as the ratio of the total area of interstitial fibrosis to the myocyte area in the entire visual field of the section.

Heart sections for immunofluorescence analysis were fixed with paraformaldehyde 4%, permeabilized using 0.2% Triton X100 in Phosphate Buffer Solution (PBS) 30 minutes at room temperature, blocked with PBS-T (0.2% Triton X100, 10% goat serum, 0.2% BSA in PBS) for 1 hour, and incubated with primary antibodies diluted in PBS-T overnight at 4° C. To evaluate cell apoptosis (day 3 post-MI), immunofluorescence analyses were performed using a TUNEL assay kit (Roche Diagnostis, Meylan, France) according to manufacturer's instructions. The digital images of immunofluorescence were acquire with a Zeiss Axioimager Z2 Apotome. And examined using ImageJ64.

Quantitative real-time PCR. Quantitative real-time PCR was performed on a Step-one Plus (Applied Biosystems) qPCR machine. GAPDH was used to normalize gene expression. The following primer sequences were used: GAPDH: Forward 5′-CGTCCCGTAGACAAAATGGTGAA-3′ (SEQ ID NO: 2), Reverse 5′-GCCGTGAGTGGAGTCATACTGGAA-CA-3′(SEQ ID NO: 3); GRZB: Forward 5′-GTGCGGGGGACCCAAAGACCAAAC-3′ ((SEQ ID NO: 4), Reverse: 5′-GCACGTGGAGGTGAACCATCCTTATAT-3′ (SEQ ID NO: 5); IL1β: Forward 5′-GAAGAGCCCATCCTCTGTGA-3′(SEQ ID NO: 6), Reverse 5′-GGGTGTGCCGTCTTTCATTA-3′(SEQ ID NO: 7); IL6: Forward 5′-TGACAACCACGGCCTTCCCTA-3′(SEQ ID NO: 8), Reverse: 5′-TCAGAATTGCCATTGCACAACTCTT-3′(SEQ ID NO: 9); IL10: Forward 5′-ACTTCCCAGTCGGCCAGAGCCACAT-3′(SEQ ID NO: 10), Reverse: 5′-GATGACAGCGCCTCAGCCGCATCCT-3(SEQ ID NO: 11); IL15: Forward 5′-CCGGTGCCAAGATCTGTGTCTCT-3′(SEQ ID NO: 12); Reverse: 5′-GTTGCACAGGGGAGTCTGGTCTT-3′((SEQ ID NO: 13); TNF-α: Forward 5′-GATGGGGGGCTTCCAGAACT-3′(SEQ ID NO: 14), Reverse 5′-GATGGGGGGCTTCCAGAACT-3′(SEQ ID NO: 15) ; MMP9: Forward 5′-GCGTCATTCGCGTGGATAAGGAGT-3′(SEQ ID NO: 16), Reverse 5′-GTAGCCCACGTCGTCCACCTGGTT-3(SEQ ID NO: 17)′

Population of patients with acute MI. The population selection methods of the French registry of Acute ST-elevation and non-ST-elevation Myocardial Infarction (FAST-MI) have been described in detail in previous publications [15]. Briefly, all patients ≥18 years of age were included in the registry if they had elevated serum markers of myocardial necrosis higher than twice the upper limit of normal for creatine kinase, creatine kinase-MB or elevated troponins, and either symptoms compatible with acute MI and/or electrocardiographic changes on at least two contiguous leads with pathologic Q waves (≥0.04 sec) and/or persisting ST elevation or depression >0.1 mV. The time from symptom onset to intensive care unit admission had to be <48 h. Patients were managed according to usual practice; treatment was not affected by participation in the registry. Of the 374 centers in France that treated patients with acute MI at that time, 223 (60%) participated in the registry. Among these, 100 centers recruited 1029 patients who contributed to a serum bank. For the present study, 1046 samples were available for Granzyme B measurement. Written informed consent was provided by each patient. Their baseline characteristics were comparable to the overall population of the registry. More than 99% of patients were Caucasians. Follow-up data was collected by contacting the patients' physicians, the patients themselves or their family, and registry offices of their birthplace. Data obtained from one-year follow-up was >99% complete. The study was reviewed by the Committee for the Protection of Human Subjects in Biomedical Research of Saint Antoine University Hospital and the data file was declared to the Commission Nationale Informatique et Liberté. Human Granzyme B analysis was carried out using the Granzyme B Human ELISA Kit (Invitrogen, ThermoFisher), following the manufacturer's instructions.

Statistical analysis. An outcome event was defined as all-cause death during the 1-year follow-up period. The primary endpoint was defined as all-cause death and was adjudicated by a committee whose members were unaware of patients' medications and blood measurements. Continuous variables are described as mean±s.d. or median, Q1, Q3 and categorical variables as frequencies or percentages. Baseline demographic and clinical characteristics, treatment factors and therapeutic management during hospitalization were compared among patients inferior or superior to the granzyme B median level (8.9 pg/mL) using chi-square or Fisher's exact tests for discrete variables and by unpaired t-tests or Wilcoxon sign-rank tests for continuous variables. Survival curves according to the granzymee B median level are estimated using the Kaplan-Meier estimator. We used a multivariable Cox proportional-hazards model to assess the independent prognostic value of variables with the primary endpoint during the 1-year follow-up period. The multivariable model comprised sex, age, body mass index, current smoking, family history of coronary disease, history of hypertension, hypercholesterolemia, previous myocardial infarction, previous stroke or transient ischemic attack (TIA), heart failure, renal failure, diabetes, Killip class, left ventricular ejection fraction, STEMI or reperfusion, hospital management (including reperfusion therapy, coronary artery bypass surgery, statins, beta blockers, clopidogrel, diuretics, low molecular weight heparin, GPIIb/IIIa inhibitors). Results are expressed as hazard ratios for Cox models with 95% confidence intervals (CIs). All statistical tests were two-sided and performed using SAS software version 9.4.

Results:

Global Granzyme B Deficiency Limits Cardiac Damage After Acute MI.

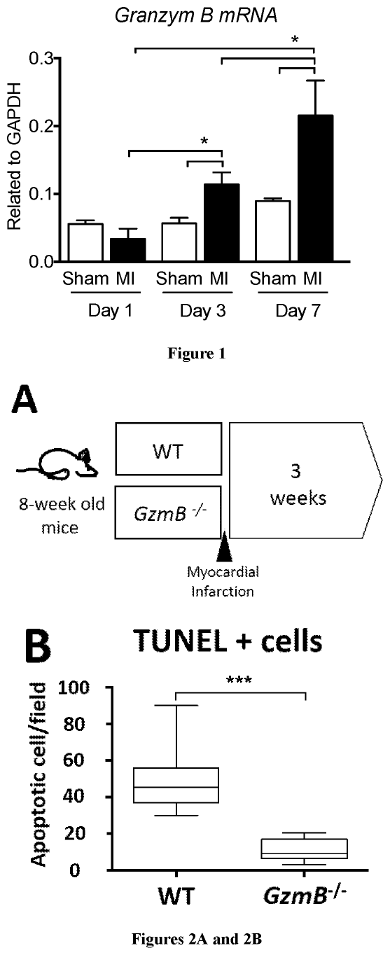

We previously showed that depletion of CD8+ T cells would be suitable for the treatment of MI (WO2017/064034). We next assessed the potential mechanisms involved in CD8⁺ T cell-mediated effects on cardiac remodeling and function. In particular, these findings prompted us to investigate the direct cytotoxic role of Granzyme B in post-ischemic cardiac remodeling. In the context of MI, Granzyme B content in the ischemic myocardium increases during the first week following coronary artery occlusion (FIG. 1).

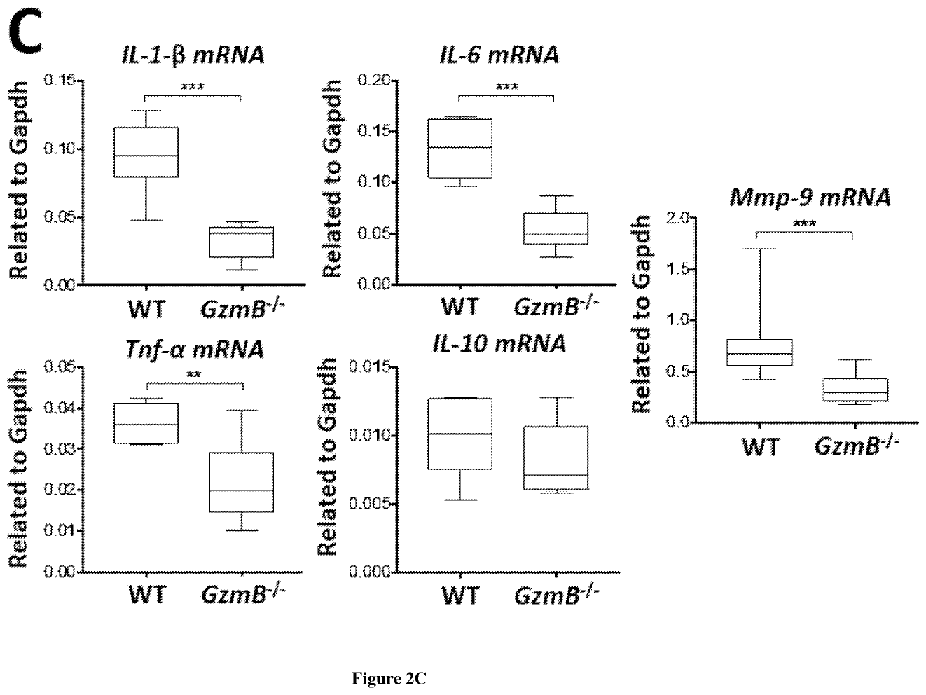

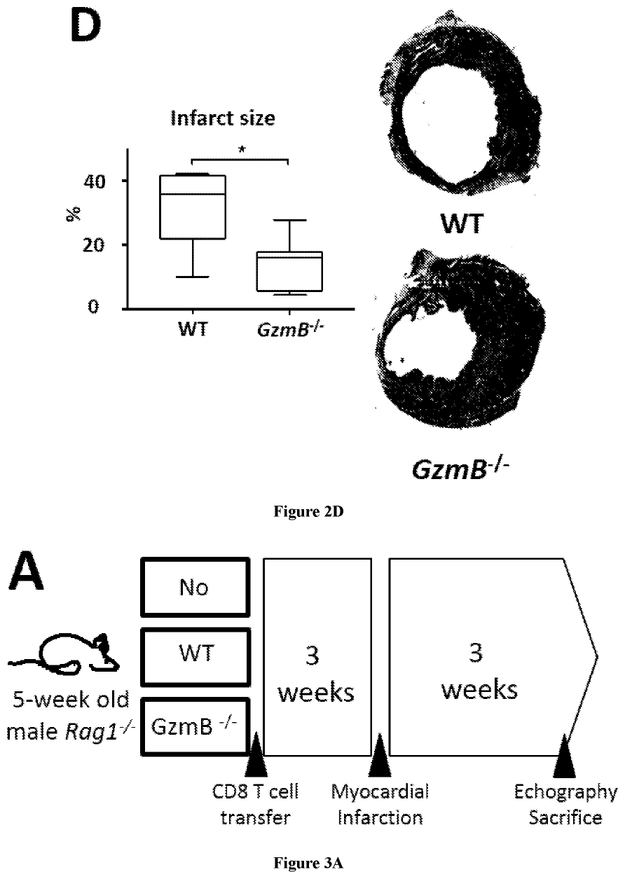

MI was induced in C57bl6 Wild type and Granzyme B-deficient (GzmB^{-/-}) adult mice (FIG. 2A). Granzyme B deficiency was confirmed by immunostaining in the spleen of GzmB^{-/-} mice (data not shown) as well as in the heart (data not shown). Following acute MI, we observed a significant reduction of TUNEL+ apoptotic cells within the injured myocardium (P<0.001) (FIG. 2B & data not shown) in peri-infarct hearts of GzmB^{-/-} mice compared to the WT control group as well as a local reduction of Il-1β, Il-6, Tnf-α and Mmp9 mRNA levels (P<0.01) (FIG. 2C). Finally, following MI, 21-day survival trends to be higher in Granzyme B deficient animals (88% vs 64%, P=0.12) (data not shown) associated with a strong reduction of infarct size (−55%, P<0.05) (FIG. 2D). These experiments suggest that Granzyme B by itself may have direct cytotoxic activity on cardiomyocytes. To confirm such a hypothesis, mouse cardiomyocytes were co-cultured in vitro with purified splenic CD8+ T cells during 24 hours. Next, T cells were removed and cardiomyocyte apoptosis was monitored during additional 48 hours using caspase-3 fluorescent dye (data not shown). Pre-incubation with activated CD8⁺ T cells (data not shown) induced cardiomyocyte apoptosis whereas non-activated CD8⁺ T cells had no pathogenic effect. Pro-apoptotic activity of activated CD8³⁰ T cells is dose-dependent and was strongly reduced in case of Granzyme B deletion (data not shown).

Granzyme B-Deficient CD8+ T lymphocytes fail to affect cardiac remodeling and function after acute MI

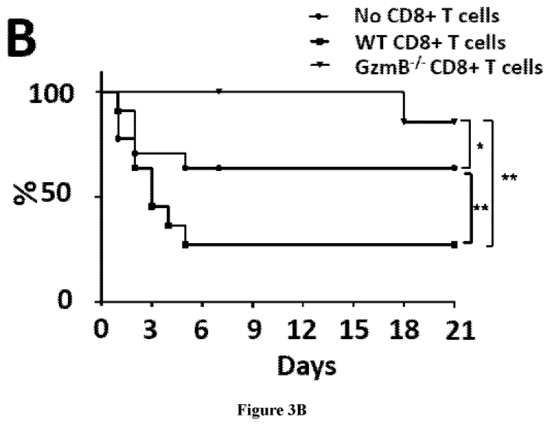

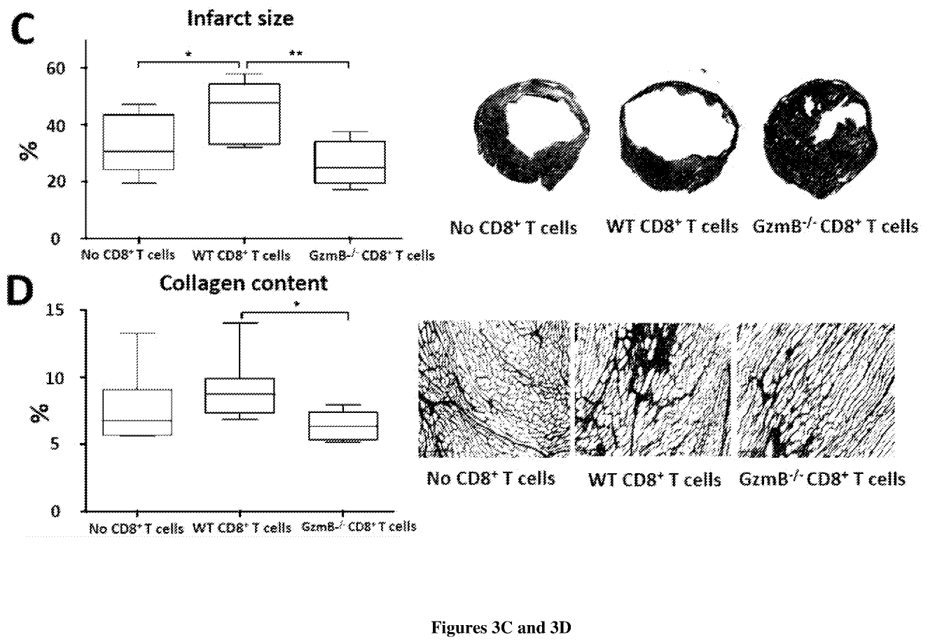

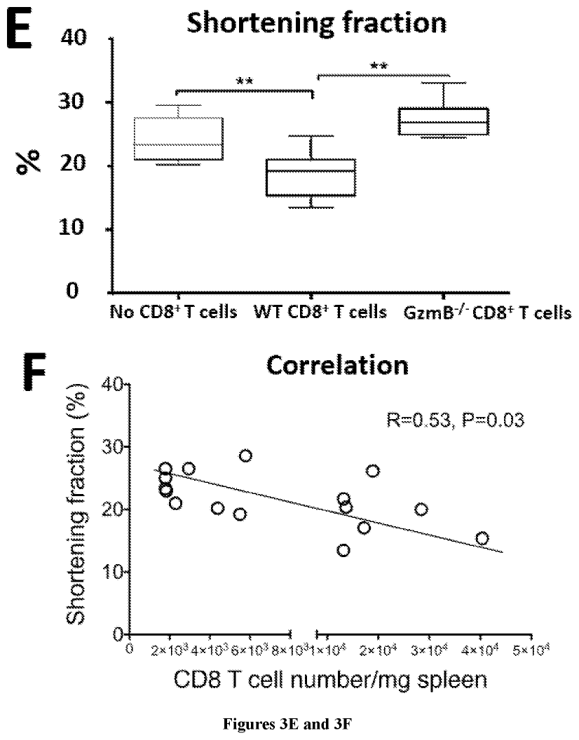

To further substantiate the role of CD8 cell-derived Granzyme B in this setting, we injected Rag1^{-/-} mice either with CD8⁺ T cell-depleted splenocytes, CD8⁺ T-depleted splenocytes re-supplemented with wild-type or GzmB^{-/-} CD8⁺ T lymphocytes (FIG. 3A). We first verified that re-supplementation with wild-type or GzmB^{-/-} CD8⁺ T lymphocytes significantly increased CD8⁺ T cell numbers in spleens and hearts of Rag1^{-/-} mice compared to mice injected with CD8⁺ T cell-depleted splenocytes only (data not shown). Interestingly, we found that re-supplementation with wild-type CD8⁺ T lymphocytes was associated with an increase of Cd11b⁺Ly6G⁻Ly6C^{hi} monocyte and CD3⁻Ly6G⁻F4/80⁺ macrophage numbers within the injured myocardium compared to the group receiving CD8+ T cell-depleted splenocytes only (data not shown), a phenotype that was abrogated in mice re-supplemented with GzmB^{-/-} CD8⁺ T lymphocytes (data not shown). Thus, in this model of re-supplementation in immunodeficient mice, CD8⁺ T cell-derived Granzyme B may be involved in selective tissue recruitment of classical monocytes and macrophages after acute MI.

We then examined the consequences of Granzyme B deficiency in CD8+ T lymphocytes on post-ischemic cardiac remodeling. We found that transfer of wild-type CD8+ T cells into Rag1^{-/-} mice reduced 21-day survival (FIG. 3B) and left ventricular shortening fraction (FIG. 3C) (p<0.05) after MI compared to the transfer of CD8-depleted splenocytes. The higher CD8 repopulation was, the worst the LV systolic dysfunction (FIG. 3F) This pathogenic effect on mortality and LV systolic function was abrogated after re-supplementation with GzmB^{-/-}CD8⁺ T lymphocytes (FIG. 3B-3C). CD8⁺ T cell supplementation also increased infarct size (p=0.04; FIG. 3C) and collagen content (FIG. 3D), which was antagonized by re-supplementation with GzmB^{-/-} CD8⁺ T lymphocytes (FIGS. 3E-3F).

Granzyme B and CD8⁺ T Cells in Human MI Setting

In human heart biopsies obtained from acute MI patients, we detected CD8⁺ T cell infiltration in the ischemic heart tissue at day 3 and Day 8 after MI (data not shown). Granzyme B is mainly detected in infarct area within the first week of MI but predominate in the peri-infarct region after Day 7 (data not shown).

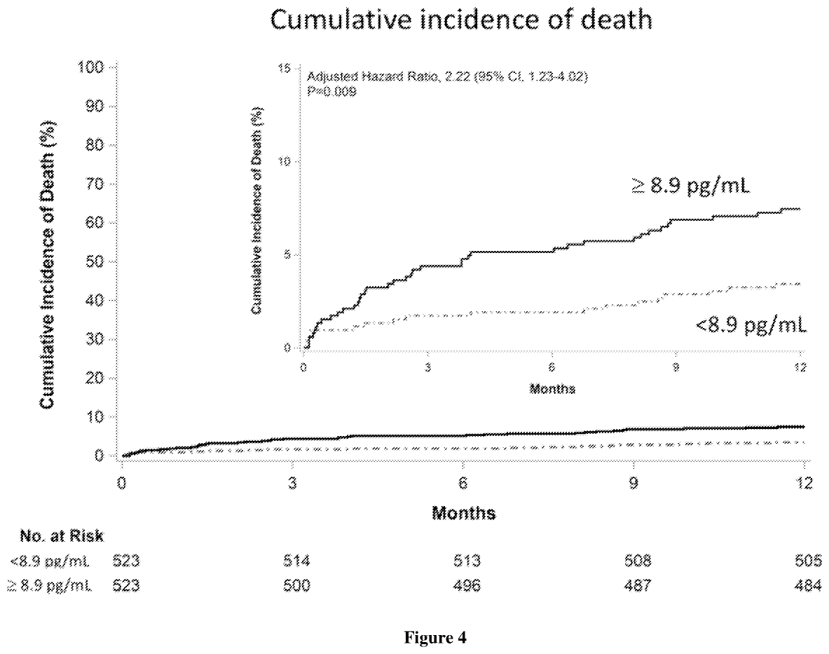

Finally, we addressed the relevance of these findings to the human disease by assessing the relationship between circulating Granzyme B levels and clinical outcomes in a cohort of 1046 patients admitted for acute MI. Patients' characteristics are given in Table 1. Interestingly, we found that patients with high circulating levels of Granzyme B (>median 8.9 pg/mL) at their admission for acute MI were at substantially increased risk of death after one year of follow-up compared to patients with low levels even after adjustment for several multivariable risk factors (hazard ratio, HR=2.2, 95% CI=1.2-4.0, p=0.009) (FIG. 4).

Discussion:

We focused here on the role of Granzyme B because it was detected in the peri-infarct area at early time points following coronary occlusion and mostly co-localized with both infiltrating CD8⁺ T cells and TUNEL+ cells. Moreover, Granzyme B has been previously identified as a major toxic protein in auto-immune diseases such as diabetes [16] as well as in inflammatory diseases including stroke [17]. We found that global Granzyme B deficiency (GzmB^{-/-} mice) is protective in the context of murine acute MI, limiting cardiac cell apoptosis, pro-inflammatory local signature and infarct size. To confirm Granzyme B-mediated cytotoxic effect of CD8⁺ T cells in the context of acute myocardial ischemia we performed additional experiments. In vitro, we showed that activated purified CD8⁺ T cells induce cardiomyocyte apoptosis and cell death was abolished when CD8⁺ T cells where isolated from GzmB^{-/-} mice. Furthermore, deleterious cardiac impact of CD8⁺ T cell reconstitution in Rag1^{-/-} mice was abolished when animals were repopulated with GzmB^{-/-} CD8⁺ T cells. Granzyme B-expressing T cells were detected in human heart tissue of MI patients mostly located in the infarct area within the first week after MI but accumulating in the border zone after day 7. Finally, we found that high plasma levels of Granzyme B within 48 hours of admission in a cohort of MI patients is associated with significantly higher 1-year mortality. Accordingly, the results prompt us to consider that neutralization of Granzyme B would be suitable for the treatment of MI.

TABLE 1
Characteristics of included patients according to baseline plasma
Granzyme B level. CAD, Coronary Artery Disease;
PCI, Percutaneous coronary intervention;
CABG, Coronary By-Pass Graft; TIA, Transient ischemic attack;
STEMI, ST Elevation Myocardial Infarction.

| | <8.9 pg/mL | ≥8.9 pg/mL | |
|---|---|---|---|
| | (N = 523) | (N = 523) | p† |
| Demographic and risk factors | | | |
| Male Sex, No (%) | 402 (76.9) | 388 (74.2) | 0.31 |
| Age, yr^{‡} | 63.0 ± 13.4 | 64.4 ± 14.0 | 0.11 |
| Hypertension, No (%) | 266 (50.9) | 276 (52.8) | 0.54 |
| Hypercholesterolemia, No (%) | 231 (44.2) | 229 (43.8) | 0.90 |
| Diabetus mellitus, No (%) | 111 (21.2) | 98 (18.7) | 0.31 |
| Family history of CAD, No (%) | 152 (29.1) | 138 (26.4) | 0.33 |
| Current smokers, No (%) | 222 (42.4) | 191 (36.5) | 0.05 |
| Prior myocardial infarction, No (%) | 69 (13.2) | 77 (14.7) | 0.48 |
| Prior PCI or CABG, No (%) | 73 (14.0) | 82 (15.7) | 0.43 |
| Prior stroke or TIA, No (%) | 18 (3.4) | 22 (4.2) | 0.52 |
| Prior heart failure, No (%) | 11 (2.1) | 18 (3.4) | 0.19 |
| Chronic kidney disease, No (%) | 22 (4.2) | 18 (3.4) | 0.52 |
| Clinical presentation | | | |
| Body mass index, kg/m^{2‡} | 26.8 ± 4.6 | 26.8 ± 4.3 | 0.94 |
| Systolic blood pressure at admission, | 144.8 ± 28.7 | 146.7 ± 27.0 | 0.26 |
| mmHg^{‡} | | | |
| Heart rate at admission, beat/min^{‡} | 79.2 ± 20.4 | 77.6 ± 18.4 | 0.18 |
| STEMI, No (%) | 297 (56.8) | 269 (51.4) | 0.08 |
| STEMI and/or revascularisation, No (%) | | | 0.22 |
| No STEMI | 226 (43.2) | 254 (48.6) | |
| STEMI alone | 63 (12.0) | 55 (10.5) | |
| STEMI and revascularisation | 234 (44.7) | 214 (40.9) | |
| Killip Max >= 2, No (%) | 86 (16.4) | 99 (18.9) | 0.29 |
| GRACE score^{‡} | 136.8 ± 34.3 | 137.5 ± 34.6 | 0.76 |
| Left ventricular ejection fraction, %^{‡} | 51.6 ± 10.5 | 52.5 ± 11.2 | 0.17 |
| Baseline biological exams | | | |
| CRP, mg/L* | 5.0 [3.0; 9.8] | 5.0 [3.0; 10.7] | 0.78 |
| In-hospital Management | | | |
| PCI, No (%) | 420 (80.3) | 399 (76.3) | 0.12 |
| Thrombolyse, No (%) | 45 (8.6) | 41 (7.8) | 0.65 |
| Coronary artery bypass surgery, No (%) | 13 (2.5) | 27 (5.2) | 0.02 |
| Statins, No (%) | 475 (90.8) | 464 (88.7) | 0.26 |
| Beta-blockers, No (%) | 431 (82.4) | 432 (82.6) | 0.94 |
| Calcium channel blockers, No (%) | 125 (23.9) | 132 (25.2) | 0.62 |
| ACE inhibitors or ARB, No (%) | 226 (43.2) | 244 (46.7) | 0.26 |
| Nitrated derivatives, No (%) | 251 (48.0) | 256 (48.9) | 0.76 |
| Aspirin, No (%) | 502 (96.0) | 513 (98.1) | 0.04 |
| Clopidogrel, No (%) | 403 (77.1) | 410 (78.4) | 0.60 |
| Heparin, No (%) | 231 (44.2) | 235 (44.9) | 0.80 |
| Low Molecular Weight Heparin, No (%) | 293 (56.0) | 306 (58.5) | 0.42 |
| Diuretics, No (%) | 137 (26.2) | 157 (30.0) | 0.17 |
| Glycoprotein IIb/IIIa inhibitors, No (%) | 231 (44.2) | 175 (33.5) | 0.0004 |
| Digitalis glycosides, No (%) | 3 (0.6) | 4 (0.8) | 1 |

†p is given by unpaired Student t or Wilcoxon rank-sum (continuous variables) and exact Pearson X² or Fisher exact test (categorical variables)
^{‡}Mean ± sd, *Median, Q1, Q3

Throughout this application, various references describe the state of the art to which this invention pertains. The disclosures of these references are hereby incorporated by reference into the present disclosure.

### REFERENCES

1. Frangogiannis N G. The inflammatory response in myocardial injury, repair, and remodelling. Nat Rev Cardiol. 2014;11: p 255-265.
2. Rosamond W D, Chambless L E, Heiss G, Mosley T H, Coresh J, Whitsel E, Wagenknecht L, Ni H, and Folsom A R. Twenty-two-year trends in incidence of myocardial infarction, coronary heart disease mortality, and case fatality in 4 US communities, 1987-2008. Circulation. 2012;125: p 1848-1857.
3. Puymirat E, Simon T, Steg P G, et al. Association of changes in clinical characteristics and management with improvement in survival among patients with ST-elevation myocardial infarction. JAMA. 2012;308: p 998-1006.
4. Lavoie L, Khoury H, Welner S, and Briere J B. Burden and Prevention of Adverse Cardiac Events in Patients with Concomitant Chronic Heart Failure and Coronary Artery Disease: A Literature Review. Cardiovasc Ther. 2016;34: p 152-160.

5. Shah A M and Mann D L. In search of new therapeutic targets and strategies for heart failure: recent advances in basic science. Lancet. 2011;378: p 704-712.
6. Yellon D M and Hausenloy D J. Myocardial reperfusion injury. N Engl J Med. 2007;357: p 1121-1135.
7. Nahrendorf M. Myeloid cell contributions to cardiovascular health and disease. Nat Med. 2018;24: p 711-720.

8. Silvestre J S, Smadja D M, and Levy B I. Postischemic revascularization: from cellular and molecular mechanisms to clinical applications. Physiol Rev. 2013;93: p 1743-1802.
9. Hofmann U and Frantz S. Role of lymphocytes in myocardial injury, healing, and remodeling after myocardial infarction. Circ Res. 2015;116: p 354-367.
10. Weirather J, Hofmann U D, Beyersdorf N, Ramos G C, Vogel B, Frey A, Ertl G, Kerkau T, and Frantz S. Foxp3+ CD4+ T cells improve healing after myocardial infarction by modulating monocyte/macrophage differentiation. Circ Res. 2014;115: p 55-67.

11. Santos-Zas I, Lemarie J, Tedgui A, and Ait-Oufella H. Adaptive Immune Responses Contribute to Post-ischemic Cardiac Remodeling. Front Cardiovasc Med. 2018;5: p 198.

12. Matzinger P. The danger model: a renewed sense of self. Science. 2002;296: p 301-305.

13. Anzai A, Anzai T, Nagai S, et al. Regulatory role of dendritic cells in postinfarction healing and left ventricular remodeling. Circulation. 2012;125: p 1234-1245.
14. Zouggari Y, Ait-Oufella H, Bonnin P, et al. B lymphocytes trigger monocyte mobilization and impair heart function after acute myocardial infarction. Nat Med. 2013;19: p 1273-1280.
15. Simon T, Verstuyft C, Mary-Krause M, Quteineh L, Drouet E, Meneveau N, Steg PG, Ferrieres J, Danchin N, and Becquemont L. Genetic determinants of response to clopidogrel and cardiovascular events. N Engl J Med. 2009;360: p 363-375.
16. Thomas H E, Trapani J A, and Kay T W. The role of perforin and granzymes in diabetes. Cell Death Differ. 2010;17: p 577-585.
17. Mracsko E, Liesz A, Stojanovic A, Lou W P, Osswald M, Zhou W, Karcher S, Winkler F, Martin-Villalba A, Cerwenka A, and Veltkamp R. Antigen dependently activated cluster of differentiation 8-positive T cells cause perforin-mediated neurotoxicity in experimental stroke. J Neurosci. 2014;34: p 16784-16795.

1.-8. (canceled)

9. A method for providing cardioprotection in a subject who experienced a myocardial infarction, said method comprising administering the subject with a therapeutically effective amount of a granzyme B inhibitor.

10. The method according to claim 9, wherein said method is suitable for reducing the risk or progression of heart failure.

11. The method according to claim 9, wherein the granzyme B inhibitor is a neutralizing antibody.

12. The method according to claim 9, wherein the granzyme B inhibitor is a monoclonal antibody.

13. The method according to claim 9, wherein the granzyme B inhibitor is an inhibitor of granzyme B expression.

14. The method according to claim 13, wherein the inhibitor of granzyme B expression is a siRNA, an antisense oligonucleotide, or a ribozyme.

15. A method of screening a test compound suitable for providing cardioprotection in a subject who experienced a myocardial infarction, said method comprising:
i) providing a test compound, and
ii) determining the ability of said test compound to inhibit the expression or activity of granzyme B.

16. A pharmaceutical composition comprising a granzyme B inhibitor.


SEQUENCE LISTING (17 sequences)

SEQ ID NO: 1 - protein, 247 aa, Homo sapiens
MQPILLLLAFLLLPRADAGEIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 2 - DNA, 23 bp, Artificial Sequence
  GAPDH Forward
cgtcccgtagacaaaatggtgaa

SEQ ID NO: 3 - DNA, 26 bp, Artificial Sequence
  GAPDH Reverse
gccgtgagtggagtcatactggaaca

SEQ ID NO: 4 - DNA, 24 bp, Artificial Sequence
  GRZB Forward
gtgcgggggacccaaagaccaaac

SEQ ID NO: 5 - DNA, 27 bp, Artificial Sequence
  GRZB Reverse
gcacgtggaggtgaaccatccttatat

SEQ ID NO: 6 - DNA, 20 bp, Artificial Sequence
  IL1beta Forward
gaagagcccatcctctgtga

SEQ ID NO: 7 - DNA, 20 bp, Artificial Sequence
  IL1 beta Reverse
gggtgtgccgtctttcatta

SEQ ID NO: 8 - DNA, 21 bp, Artificial Sequence
  IL6 Forward
tgacaaccacggccttcccta

SEQ ID NO: 9 - DNA, 25 bp, Artificial Sequence
  IL6 Reverse
tcagaattgccattgcacaactctt

SEQ ID NO: 10 - DNA, 25 bp, Artificial Sequence
  IL10 Forward
acttcccagtcggccagagccacat

SEQ ID NO: 11 - DNA, 25 bp, Artificial Sequence
  IL10 Reverse
gatgacagcgcctcagccgcatcct

SEQ ID NO: 12 - DNA, 23 bp, Artificial Sequence
  IL15 Forward
ccggtgccaagatctgtgtctct

SEQ ID NO: 13 - DNA, 23 bp, Artificial Sequence
  IL15 Reverse
gttgcacaggggagtctggtctt

SEQ ID NO: 14 - DNA, 20 bp, Artificial Sequence
  TNF-alpha Forward
gatggggggcttccagaact

SEQ ID NO: 15 - DNA, 20 bp, Artificial Sequence
  TNF-alpha Reverse
gatggggggcttccagaact

SEQ ID NO: 16 - DNA, 24 bp, Artificial Sequence
  MMP9 Forward
gcgtcattcgcgtggataaggagt

SEQ ID NO: 17 - DNA, 24 bp, Artificial Sequence
  MMP9 Reverse
gtagcccacgtcgtccacctggtt